(19) United States
(12) Patent Application Publication
(10) Pub. No.: US 2002/0106658 A1
(43) Pub. Date: Aug. 8, 2002

(54) Blocking chemistries for nylon membrane

(76) Inventors: Ian Wells, Escondido, CA (US); Ing Ching Lin, North Haven, CT (US); Todd E. Arnold, Glastonbury, CT (US); Mark T. Meyering, Middlefield, CT (US)

Correspondence Address: Cummings & Lockwood, P.O. Box 1960, New Haven, CT, 06509-1960, US

(21) Appl. No.: 09/900,349

(22) Filed: Jul. 6, 2001

Related U.S. Application Data
(60) Provisional application No. 60/220,023, filed on Jul. 21, 2000.
(60) Provisional application No. 60/217,020, filed on Jul. 11, 2000.

(51) Int. Cl. C12Q 1/68; G01N 33/53; G01N 33/542; B32B 5/02

(52) U.S. Cl. 435/6; 435/7.9; 442/59

(57) ABSTRACT

The present disclosure provides an effective chemical blocking agents for use with nylon membrane suitable for use in immunodiagnostic assays, flow-through assays and nucleic acid detection assays and methods of preparing and using same and to effective chemical blocking agents for treating unreinforced and reinforced nylon membrane and methods for the preparation thereof and the application thereof to unreinforced and reinforced nylon membrane, the chemical blocking agent including an effective amount of alkaline treated casein; an effective amount of polymer; and an effective amount of surfactant.

### RELATED APPLICATIONS

This application is a continuation-in-part of commonly owned U.S. Provisional Patent Application Serial No. 60/220,023, filed Jul. 21, 2000, of Wells et al., entitled “Improved Blocking Chemistries For Nylon membrane,” and is related to commonly owned U.S. Provisional Patent Application Serial No. 60/217,020, filed Jul. 11, 2000, of Lin, entitled “Membrane And Method For Making Same With Consistent Wicking Properties For Lateral Flow Assays,” the disclosure of each is herein incorporated by reference to the extent not inconsistent with the present disclosure.

### BACKGROUND OF THE DISCLOSURE

The present disclosure relates to effective chemical blocking agents for use with nylon membrane suitable for use in immunodiagnostic assays, flow-through assays and nucleic acid detection assays and methods of preparing and using same and, more particularly, to effective chemical blocking agents for use with reinforced and unreinforced nylon microporous membrane having lateral flow properties useful in immunodiagnostic assay applications and with unreinforced and reinforced nylon membrane for use with flow-through assays and nucleic acid detection assays and, most particularly, to effective chemical blocking agents for use with reinforced nylon microporous membrane having improved scrims that provide specific lateral flow properties such that the combination produced thereby is useful in immunodiagnostic assay applications and for use with unreinforced and reinforced nylon membrane useful for in vitro diagnostic test kits having flow-through assays and for the transfer of nucleic acids (DNA and/or RNA) to nylon membrane used in nucleic acid detection assays and to a method for using such effective chemical blocking agents with regular, unreinforced and reinforced nylon membrane for use in in vitro diagnostic test kits having flow-through assays, for use in nucleic acid detection assays and to reinforced nylon microporous membrane having improved scrims treated with the effective chemical blocking agents for use in lateral flow immunodiagnostic assay applications and unreinforced or reinforced nylon membrane treated with the effective chemical blocking agents used for flow-through assays and nucleic acid detection assays.

There has been an expanding need for the development of diagnostic devices to detect substances in the physiological test samples for patients at home and in doctor's office or other healthcare facilities without using sophisticated instruments. The advantage of lateral flow immunoassay is that the device can offer a simple, one-step analysis with accurate results within several minutes when executed by less-skilled or unskilled personnel. Typical at home and in doctor's office applications include pregnancy test and Streptococcus assay kits.

Membranes have become invaluable tools in the clinical arts. Specifically, membranes are integral to immunodiagnostic assays. However, currently available membranes possess qualities that limit their utility within the context of the foregoing applications.

Immunodiagnostic assays are generally performed by applying a test liquid containing antigens to a porous membrane containing antibodies. As the test liquid laterally diffuses through the membrane, antibodies will bind antigens to which they are directed with a high degree of specificity. The binding of the antibodies to the antigens serves as a detection means (e.g., the visualization of the presence of antigens), and the specificity with which antibodies bind to antigens allows for the determination of whether or not the test liquid contains specific antigens. Therefore, in immunodiagnostic assays, the membrane desirably possesses optimal immunodiagnostic properties. In other words, it is desirable that the membrane allow for optimal lateral diffusion of the test liquid, allow for adequate visualization of the existence of antigens in the test, allow for adequate protein binding, is hydrophilic, is capable of being uniformly manufactured in order to yield consistent results and is safe to use.

The most common types of membranes available for use in immunodiagnostic are cellulose-based membranes (e.g., nitrocellulose and cellulose acetate membranes). Both of these membranes, however, possess qualities that limit their utility in the foregoing applications. Nitrocellulose is prepared by the nitration of naturally occurring cellulose. During nitration, a broad distribution of heterogeneous oligomeric and polymeric nitrated products is produced as a consequence of the partial acid digestion of cellulose. Exacerbating the problem is the fact that the purity of the cellulose starting material depends on its source and pre-nitration treatment. Consequently, uniformity in the manufacture and in the finished product(s) of nitrocellulose membranes is difficult to achieve. For similar reasons, it is also difficult to achieve uniformity in the manufacture of other cellulose membranes, such as cellulose acetate membranes. Furthermore, nitrocellulose membranes present numerous laboratory safety concerns by virtue of their flammability and explosiveness. Cellulose acetate and nitrocellulose membranes are also disadvantageous in that such membranes are very brittle, easily broken and difficult to wet using aqueous solutions (hydrophobic).

Typically, large pore size nitrocellulose membranes with pore size from about two (2) to about twenty (20) microns are used in lateral flow immunoassay applications. As discussed above, there are many problems with using nitrocellulose membrane for lateral flow applications including, but not limited to, the fragile nature of the membrane making it difficult to handle in the manufacturing process, the laminated version of nitrocellulose membrane improves the mechanical strength, but suffers from a non-uniform wicking front, and, more importantly, nitrocellulose membrane has inconsistent properties such as wicking and protein binding due to the nature of nitrocellulose resin itself and the manufacturing process of nitrocellulose membrane.

As is known, at some point the nitrocellulose membrane or other nylon membrane should be treated to block any remaining non-specific binding sites. As taught in international publication No. WO 88/08534 entitled: “Immunoassays and Devices therefore,” the disclosure of which is hereby incorporated by reference to the extent not inconsistent with the present disclosure, blocking can be achieved by treatment with protein (e.g. bovine serum albumin or milk protein), or with polyvinyl alcohol or ethanolamine for any combination of these agents.

As shown in the subject publication, examples of flow rates obtained with various materials including nitrocellulose, polyester backed nitrocellulose showed a “time to flow” for 45 millimeter of length to vary from between 3.2 and 19.2 minutes.

Specifically, at page 34 of the referenced publication, it was stated that excess binding sites may be blocked by the addition of, for example, bovine serum albumin: 4 mls of 150 mg/ml BSA in 5 mM NaCl pH 7.4 is added to the reaction mixture and after 15 minutes incubation at room temperature, the solution is centrifuged at 3000 g for 10 minutes, and pellet resuspended in 10 mls of 0.25% (w/v) dextran/0.5% (w/v) lactose and 0.04M phosphate buffer.

Thus, it can be seen that blocking agents are recognized as being required and have been used in the prior art, however, these blocking agents have resulted in slow wicking time and in low sensitivity, especially when used on nylon membrane for which these blocking agents have not been optimized.

Another useful format for producing in vitro diagnostic test kit utilizes membranes and blocking chemistries in the flow-through mode, as opposed to lateral flow. Flow-through assays are used for a variety of diagnostic tests, using reagents similar to those used in lateral flow assays. Both “sandwich” assays and “competitive” assays have been produced. Flow-through assays are generally multiple step procedures, involving more operator interaction, when compared to lateral flow assays. Nylon would be a preferred substrate for these flow-through test assays, due to the natural hydrophilicity of nylon, and nylon's ability to selectively immobilize one or more of a variety of test components as needed, if effective chemical blocking agents are used.

With the nylon membrane substrates used in flow-through type tests, it is more important to have uniform and controlled trans-membrane flow-through attributes, which are largely controlled by the uniformity of pore size and the selection of reinforcements which are uniform and non-restrictive to trans-membrane flow rates, than in lateral flow assay applications. In other words, a more traditional nylon microporous filtration membrane having a pore size and pore distribution optimized for trans-membrane wicking flow rate is more likely to be useful than one optimized for lateral flow attributes. Thus, such flow-through nylon membrane assays require chemical blocking agents optimized for the specific nylon microporous filtration membrane used in the specific diagnostic kit to enhance the performance and sensitivity, and to potentially reduce the complexity of the flow through membrane assay.

Additionally, as is known, transfer of nucleic acids (DNA and/or RNA) to nylon membrane supports after resolution through agarose or acrylamide gels is a common method for gene discovery analysis. During the transfer, nucleic acids are treated so they become single stranded allowing complementary sequences (probes) to hybridize to appropriate sequences in the nucleic acid bound to the nylon membrane. The transfer of DNA to nylon, or other types of membrane, is known as a Southern Blot, after the inventor of the procedure, E. M. Southern. The transfer of RNA is termed a Northern Blot. Once transferred to the membrane the nucleic acid is irreversibly bound to the nylon membrane by means of complete drying in the presence of heat, or by exposing the membrane to ultraviolet irradiation.

Other methods for applying nucleic acids to membranes exist. Slot or dot blots, where nucleic acid samples are placed in “wells” in a vacuum manifold, which contains the nylon membrane below the wells. Suction is applied and the nucleic acid is transferred to the membrane where it is subsequently fixed to the membrane as described above.

Neither of the above methods is amenable to massively parallel nucleic acid analysis. To address these needs, high-density arrays are employed. In these techniques, nucleic acids are applied to nylon membranes or modified glass surfaces. Two scales exist in array production: Macroarrays, which typically use 22×22 cm sheets of nylon membrane to spot down PCR products in low microliter/high nanoliter amounts and microarrays which use silanized or otherwise modified glass microscope slides for spotting samples of low nanoliter to picoliter volumes.

In the above methods, detection of the nucleic acids bound to the solid support is achieved either directly or indirectly. In direct detection, a complementary sequence of nucleic acid that hybridizes to a specific region of the solid phase bound nucleic acid, is radioactively labeled or fluorescently labeled. Such labeled complementary molecules, when bound to the target sequence can be detected without further manipulation. In the second indirect detection method, the complementary sequence is modified with biotinylated nucleotides, other steroid hapten-coupled nucleotides, or enzyme-labeled DNA sequences. Each of these systems requires that a second reagent interact with the bound nucleic acid: modified complement hybrid molecule to allow for visualization of a signal.

One attribute in making nylon membrane highly attractive as a solid support for blotting and arraying applications is nylon's ability to bind avidly nucleic acids. However, this characteristic can also pose significant problems when attempting to detect specifically a particular nucleic acid molecule that is bound to the nylon membrane. Nucleic acids bound to nylon membranes are detected with a complementary molecule(s) using radioisotopic, chemiluminescent, chemifluorescent, and/or fluorescent methods. Unfortunately, nylon has a high affinity for these molecules used to detect the bound nucleic acids, especially for proteins, steroid, haptens, and fluorophore-containing nucleic acids. To address this problem, application specific blocking reagents that prevent interaction between the nylon itself and these labeling and detection reagents are available from distributors who sell reagent kits based on the aforementioned non-isotopic techniques. However, these blocking reagents appear only to work with a specific kit format.

It is believed that these currently available blocking reagents do not function across all non-isotopic and isotopic detection methods. In fact, unacceptable levels of background signal result from the non-specific interaction between the macromolecules specifically directed toward a membrane-bound nucleic acid and the nylon membrane itself. Such non-specific binding is undesirable as it leads to unacceptable levels of background signal (for example, splotching, smearing, “leopard-spotting”) on the blot or array. Such non-specific binding also reduces sensitivity and makes discrimination of “positive” bands or spots from “false positive” bands or spots intractable.

Examples exist which utilize nylon membrane in these assays but background signal problems (for example, splotching, smearing, “leopard-spotting”) on the blot or array has remained problematic, even with the blocking buffers currently available from distributors of these detection methodologies. Thus, there appears to be no chemical blocking agents presently available that provide for the use of nylon membranes possessing a range of positive charge densities to be used in chemiluminescent and fluorescent-based detection methods without the above mentioned significant background signal problems.

Based on the foregoing problems with unacceptable background signal, there exists a need for effective chemical blocking agents for use with nylon membrane suitable for use in immunodiagnostic assays for lateral flow assay applications, flow-through assays and nucleic acid detection assays applications. Such chemical blocking agents should include alkaline treated casein when applied to nylon membrane used in lateral flow diagnostic applications, flow-through assays and nucleic acid detection assays applications. Such chemical blocking agents should include alkaline treated casein such that the casein surface is modified in such a way as to maintain the particle size and distribution of the casein molecules when used in lateral flow diagnostic applications, flow-through assays and nucleic acid detection assays applications. Such chemical blocking agents should include alkaline treated casein such that the casein molecules interact with the binding sites of nylon in the smallest possible form when used in lateral flow diagnostic applications, flow-through assays and nucleic acid detection assays applications. Such chemical blocking agents should more effectively block the nylon as opposed to if the casein were clumping or aggregating when used in lateral flow diagnostic applications, flow-through assays and nucleic acid detection assays applications. Such chemical blocking agents should include stabilized casein that effectively decreases the wicking time in lateral flow diagnostic applications. Such membrane treated with such chemical blocking agents should provide for the analyte of interest to be detected at a lower concentration that prior known blocking agents thereby increasing the sensitivity for specific lateral flow diagnostic applications. Such chemical blocking agents should provide for the use of nylon membranes having a variety of positive charge densities to be used in chemiluminescent and fluorescent-based detection methods.

The present disclosure provides such effective chemical blocking agents for treating unreinforced and reinforced nylon membrane and methods for the preparation thereof and the application thereof to unreinforced and reinforced nylon membrane.

These and other advantages of the present disclosure, as well as additional inventive features, will be apparent from the description of the disclosure provided herein.

### SUMMARY OF THE DISCLOSURE

An object of the present disclosure is to provide effective chemical blocking agents for use with nylon membrane suitable for use in immunodiagnostic assays for lateral flow assay applications, flow-through assays and nucleic acid detection assays applications.

Another object of the present disclosure is to provide chemical blocking agents that include an alkaline treated casein when applied to nylon membrane used in lateral flow diagnostic applications, flow-through assays and nucleic acid detection assays applications.

A further object of the present disclosure is to provide chemical blocking agents that include an alkaline treated casein such that the casein surface is modified in such a way as to maintain the particle size and distribution of the casein molecules when used in lateral flow diagnostic applications, flow-through assays and nucleic acid detection assays applications.

Yet a further object of the present disclosure is to provide chemical blocking agents that include an alkaline treated casein such that the casein molecules interact with the binding sites of nylon in the smallest possible form when used in lateral flow diagnostic applications, flow-through assays and nucleic acid detection assays applications.

Yet another object of the present disclosure is to provide chemical blocking agents that more effectively block the nylon as opposed to if the casein were clumping or aggregating when used in lateral flow diagnostic applications, flow-through assays and nucleic acid detection assays applications.

Still another object of the present disclosure is to provide chemical blocking agents that include a stabilized casein that effectively decreases the wicking time in lateral flow diagnostic applications.

Another object of the present disclosure is to provide a membrane that when treated with a chemical blocking agent which provides for the analyte of interest to be detected at a lower concentration than prior known blocking agents thereby increasing the sensitivity for specific lateral flow diagnostic applications.

A further object of the present disclosure is to provide effective chemical blocking agents for treating nylon membrane and methods for the preparation thereof and the application thereof to nylon membrane used in lateral flow diagnostic applications, flow-through assays and nucleic acid detection assays applications.

Yet a further object of the present disclosure is to provide chemical blocking agents for the use with nylon membranes having a variety of positive charge densities to be used in chemiluminescent and fluorescent-based detection methods. In accordance with these and further objects, one aspect of the present disclosure includes chemical blocking agents for use with nylon membrane suitable for use in assays comprising: an effective amount of alkaline treated casein; an effective amount of polymer; and an effective amount of surfactant.

Another aspect of the present disclosure includes a method of preparing chemical blocking agents for use with nylon membrane suitable for use in assays comprising the acts of: mixing an effective amount of potassium phosphate buffer and an effective amount of alkaline treated casein in a receptacle; adding an effective amount of surfactant; diffusing the surfactant into solution; once in solution, adding an effective amount of sucrose; dissolving the effective amount of sucrose; once the sucrose is dissolved, adding an effective amount of polymer; allowing the effective amount of polymer to dissolve for about sixty (60) minutes; upon expiration of the sixty (60) minutes, filtering the solution through an about 0.2 μm filtration device into a receptacle.

Still another aspect of the present disclosure includes a nylon microporous membrane suitable for use in assays comprising: a nylon membrane formed from a dope; and an effective amount of a blocking agent, operatively distributed throughout the nylon membrane.

Another aspect of the present disclosure includes an immunodiagnostic assay kit comprising the membrane of the present application and a means for detecting an analyte of interest.

Other objects and advantages of the disclosure will be apparent from the following description, the accompanying drawings and the appended claims.

### DETAILED DESCRIPTION OF REPRESENTATIVE EMBODIMENTS OF THE DISCLOSURE

The present innovative chemical blocking agents for use with unreinforced and reinforced nylon microporous membrane are suitable for use in lateral flow immunodiagnostic assays, flow-through assays and nucleic acid detection assays and methods of preparing and using same. The chemical blocking agents for use with the unreinforced and reinforced nylon membranes described herein, presently preferably, include alkaline treated casein. Such reinforced nylon membranes treated with the chemical blocking agent of the present application for use in immunodiagnostic assays not only provide a more consistent process but also provide a higher wicking rate and a more sensitive detection level than the nitrocellulose and other membranes presently used in immunodiagnostic assays.

As stated in the background of the disclosure of the prior provisional application that has been incorporated by reference, an object of that disclosure was to select and use a reinforcing scrim which exhibited high uniformity of properties on the scrim surface, exhibited high uniformity of thickness, exhibited high uniformity of distribution of fibers, and exhibited high uniformity of macro and micro appearance when used in reinforced microporous membrane for lateral flow diagnostic applications.

As is known in the art, reinforced membrane used in lateral flow immunodiagnostic assays applications, flow-through assays and nucleic acid detection assays are, by necessity, porous. In lateral flow assays, the desired pore size of the membrane is a function of the wicking time. The larger pore size membranes have provided faster wicking time than have the smaller pore size membranes. Preferably, the pore size of the membrane, as detected by Bubble Point techniques (examples include but are not limited to: Initial Bubble Point or Foam All Over Point), is in the range of about 01.0 micron to about 20 microns; more preferably, the pore rating is in the range of about 5.0 microns to about 15.0 microns; and most preferably, the pore rating is in the range of about 8.0 microns to about 12.0 microns.

The inventive reinforced membrane, as described in the incorporated by reference provisional application, can be used within the context of any application where it is desired to detect an analyte of interest. While the membrane can be used in any suitable way, preferably, the method for using that inventive membrane containing the new, improved chemical blocking agents comprises: contacting the membrane with a fluid comprising the analyte of interest, allowing the fluid to laterally flow by capillary action through the membrane, and detecting the analyte of interest on the membrane.

Another embodiment of the present disclosure is an immunodiagnostic assay kit, which can be used for IVD assays. The immunodiagnostic assay kit comprises a reinforced membrane, as disclosed in the incorporated by reference provisional application, containing the new, improved chemical blocking agents and a means for detecting an analyte of interest. While any suitable detection means can be utilized within the context of the present disclosure, the detection means is presently preferably a colloidal metal, colloidal gold, colored liposomes, colored polymeric beads, polymerized dye molecule, or other visualization-aiding substance which can be conjugated with an analyte-specific detection molecule.

As previously stated, optimal immunodiagnostic properties include a membrane's ability to be safely used in a laboratory environment (e.g. the membrane is not flammable or explosive), its ability to be uniformly manufactured in order to yield consistent experimental results and its hydrophilicity. Further, optimal properties include the membrane's ability to strongly bind analyte-specific molecules of interest. Additionally, the membrane must be able to be further treated with appropriate blocking treatment which allows free lateral passage of labeling and/or detection conjugates of analyte or signal generating moieties which, if not blocked, would result in non-specific signal (i.e. the membrane is capable of a high signal-to-noise ratio). Therefore, any membrane prepared by the foregoing preparative methods can be tested for its immunodiagnostic properties, and the process conditions of the preparative method can be adjusted in response to the test so as to enhance or otherwise alter the immunodiagnostic properties of the membrane produced therefrom.

However, while the reinforced nylon membrane incorporating the new scrim, as described in the incorporated by reference provisional application, proved particularly advantageous with respect to lateral flow/wicking rates, it remained to be seen how such improved wicking rates would hold up in immunodiagnostic assays used in actual immunodiagnostic test situations.

Specifically, since additional material needed to be incorporated into the reinforced nylon membrane, including a chemical blocking agent for blocking the non-specific binding locations in the nylon, in order to be commercially viable, additional tests needed to be conducted that replicated real commercial immunodiagnostic assays use situations. Such tests would, by necessity, need to incorporate non-specific binding blocking agents to prevent the analyte of interest from binding to the nylon prior to reaching the capture zone. In other words, the chemical blocking agent must allow the analyte of interest to traverse to the capture zone in sufficient concentration to indicate the presence or absence of the analyte of interest. Toward that end, specific tests were conducted using a new, improved blocking agent.

As a result of further development, it has now been determined that the innovative reinforced nylon microporous membrane including specific scrims when combined with the new, improved blocking agents of the present application produce outstanding results when used in lateral flow immunodiagnostic assay applications.

Specifically, new, improved chemical blocking agents have been discovered which provide for the rapid wicking on a consistent front with the liquid reaching the capture zone containing sufficient concentration of the analyte of interest to provide superior test results. This has now been accomplished by treating the innovative reinforced nylon microporous membrane with the newly discovered improved chemical blocking agents. While only reinforced nylon membranes have been tested, it should be understood that an appropriate unreinforced nylon membrane could be used with the new, improved chemical blocking agents of the present application.

First prior to giving specific examples, it is believed appropriate to describe the process involved with the transformation of the reinforced nylon membrane, as described in the prior provisional application, into commercial lateral flow immunodiagnostic assays.

Once the reinforced nylon membrane is manufactured into rolls, as described in the provisional application, the lateral flow immunodiagnostic assays manufacturing sequence typically begins when a roll of the membrane is received at the lateral flow immunodiagnostic assays manufacturing facility. There, the roll is unrolled and as the roll is being unrolled, the roll is typically simultaneously “striped,” i.e. a pattern is applied containing the specific capture chemistry or chemistries to form the capture zone and to form the end point chemistry, the endpoint being the area of the membrane that indicates that a specific test has been completed. Thus, typically, two stripes of chemicals are applied on the membrane roll as it is unrolled. It is possible to add even more stripes, which would indicate for example, positive control or negative control for the test.

As is known, many of the tests, for example the pregnancy test, have either a plus sign that develops or a minus sign that develops in one window and then a second window at the top of the wicking material also changes color to indicate completion of the test. However, other commercial lateral flow immunodiagnostic assay tests do not use a second window to indicate completion of the test.

After the capture zone chemistry and the end zone chemistry, if used, have been applied to the reinforced nylon membrane, the membrane having the appropriate chemistry applied thereto is dried on the membrane, as is known in the art, such as, for example, either by conventional drying methods or by application of UV light or equivalent process. Once the nylon membrane roll has had the appropriate chemistry operatively applied thereto, the nylon membrane roll is unwound and dipped into the chemical blocking agent solution and any excess chemical blocking agent solution is removed by scraping or blotting prior to the saturated nylon membrane roll being dried, such as, for example, in an oven, as is known in the art.

Once the above had been accomplished, the chemical blocking agent including an alkaline treated casein of the present application is applied thereto and then dried. It is believed that, in the course of drying, the chemical blocking agents including the alkaline treated casein remain functional so that, when dried on the nylon membrane, the chemical blocking agent including the alkaline treated casein is in the correct positions to prevent or block nonspecific binding such that greater concentrations of the analyte of interest reach the capture zone. Specifically, after the drying process the alkaline treated casein needs to be functionally present on and within the nylon membrane after drying.

Once the above has been accomplished, the nylon membrane roll is cut into strips and the strips are assembled into the test housing, such as, for example, plastic housing, as is known in the art.

Now we turn our attention to the formulation and preparation of the new and improved chemical blocking agents that have been found to be effective in lateral flow immunodiagnostic assay applications when used with the reinforced nylon membrane described above and in the previously mentioned provisional patent application. Two specific formulations have been found to be especially effective in lateral flow immunodiagnostic assay applications. It should be understood that, while these formulations have been found to be effective, they are merely representative of any one of a plurality of possible formulations which could be operative to block the non-specific binding sites of nylon membrane. It should be understood that these formulations are also believed to be effective for use with reinforced and nonreinforced nylon membrane when used for flow-through assays and nucleic acid detection assays as well.

Conjugate Matrix & Membrane Blocking Buffer A

Formulation A comprising:

about 0.15% Alkaline treated Casein+about 0.25% Polyvinyl alcohol (PVA) (9,000-10,000 M.W.)+about 0.1% Surfactant 10G+about 25 mM Potassium phosphate+about 0.12% Boric acid+about 0.05% Sodium azide+about 0.015% Sucrose.

Conjugate Matrix & Membrane Blocking Buffer B

Formulation B comprising:

About 0.2% Alkaline treated Casein+about 0.25% Polyvinyl alcohol (PVA) (9,000-10,000 M.W.)+about 0.2% Surfactant 10G+about 25 mM Potassium phosphate+about 0.12% Boric acid+about 0.05% Sodium azide+about 0.015% Sucrose.

Conjugate Matrix & Membrane Blocking Buffer C

Formulation C comprising:

About 0.15% Alkaline treated Casein+about 0.25% Polyvinyl alcohol (PVA) (9,000-10,000 M.W.)+about 0.2% Chemal LA-9+about 25 mM Potassium phosphate+about 0.12% Boric acid+about 0.05% Sodium azide+about 0.015% Sucrose.

Conjugate Matrix & Membrane Blocking Buffer D

Formulation D comprising:

About 0.2% Alkaline treated Casein+about 0.25% Polyvinyl alcohol (PVA) (9,000-10,000 M.W.)+about 0.3% Chemal LA-9+about 25 mM Potassium phosphate+about 0.12% Boric acid+about 0.05% Sodium azide+about 0.015% Sucrose.

The above formulations have been found to work particularly well for specific lateral flow immunodiagnostic assay applications and at least one of the new and improved chemical blocking agents will be used in the examples below. However, prior to explaining the formulations of the new and improved chemical blocking agents in detail, it is believed appropriate to describe the method for making the constituent components thereof.

First, it should be pointed out that presently only Sigma style casein or casein manufactured or supplied by Sigma has been tested in the above new and improved chemical blocking agent formulations. Another style casein was tried, specifically Hammerstein casein, a different manufacturer made by a different method or prepared with a different method. However, the Hammerstein style casein, by itself without modification or incorporation into the above formulations, was not operable as a chemical blocking agent, whereas unmodified Sigma casein by itself was operable as a chemical blocking agent before being included in the formulations above. Because of the Hammerstein casein not being operable alone as a chemical blocking agent, no attempt was made to alkaline treat the Hammerstein casein. In fact, in one test, with one representative nylon membrane, with the Sigma casein alone as the chemical blocking agent, satisfactory but less than optimum results were achieved, resulting in the decision to attempt to improve the performance of the Sigma casein as a chemical blocking agent.

However, it is not intended that casein manufactured or supplied by other companies are to be excluded from the scope of this application. In fact, with further development, the Hammerstein casein might prove operable and, thus, it is specifically intended that any chemical blocking agent that includes casein that effectively, operatively blocks a high percentage of the nonspecific binding points of nylon membrane be included within the scope of this application.

First, we will describe the preparation of chemical blocking agent A and then we will describe the preparation of the constituent components that make up the reactants of chemical blocking agent A.

Preparation of Conjugate Matrix & Membrane Blocking Buffer or Chemical Blocking Agent A

The equipment required for the preparation of Chemical Blocking Agent A includes: a Balance, Weigh Boats, Glassware, Measuring Cylinders, Flasks, a Stir Plate, a Stir Bar, a Filtration Device and a pH Meter.

The chemicals required: for the preparation of Chemical Blocking Agent A includes: 50 mM Potassium Phosphate buffer, 25 mM Potassium Phosphate buffer Alkaline Treated Casein, PVA, Surfactant 10G, Sucrose and Sodium Azide (stabilizer) can be shipped at a concentration equal to or less than 0.5%

Formulation per 133 mL

| | | |
|---|---|---|
| 50 mM Potassium Phosphate Buffer | 50.0 | mL |
| 25 mM Potassium Phosphate Buffer | 33.0 | mL |
| 0.4% Alkaline Treated Casein | 50.0 | mL |
| PVA | 0.330 | gm |
| Surfactant 10 G | 0.133 | gm |
| Sucrose | 0.020 | gm |
| Sodium Azide | 0.050 | gm |

The above constituent ingredients are then combined in a clean receptacle. First, in the clean receptacle, 50 mM potassium phosphate buffer and 25 mM potassium buffer and alkaline treated casein volumes are added. Next, the receptacle containing the above reactants are placed on the stir plate, the stirrer is turned on and the stir bar is added. Then, surfactant 10G is added and allowed to diffuse into solution. Once in solution, sucrose is added and allowed to dissolve. Once the sucrose is dissolved, the PVA is added and allowed to dissolve for about sixty (60) minutes. Upon expiration of the sixty (60) minutes, the solution is filtered through an about 0.2 μm filtration device into a receptacle which is then covered and labeled with the appropriate part and lot number, the Date of manufacture, the Expiration Date prior to being stored at about five degrees C. (5° C.). Any remaining material should be discarded after expiration date.

Preparation of Conjugate Matrix & Membrane Blocking Buffer or Chemical Blocking Agent C

The only difference in the preparation of Chemical Blocking Agents A & C is that Chemical Blocking Agent C uses the Surfactant Chemal LA-9 in place of Surfactant 10G. In all other aspects, the formulation and the manufacturing process are the same.

As stated above, before the manufacture of the above-described Chemical Blocking Agents, it is necessary to prepare each of the specific ingredients contained in the above-described Membrane Chemical Blocking Agents as follows.

50 mM Potassium Phosphate Buffer pH 7.4-7.6

The equipment required for the preparation of 50 mM Potassium Phosphate Buffer pH 7.4-7.6 includes: a Glass Bottle (screw cap), Volumetric Flask, a Stir Plate, a Stir Bar and a pH meter.

The chemicals required: for the preparation of 50 mM Potassium Phosphate Buffer pH 7.4-7.6 includes: 0.5 M Dibasic Potassium Phosphate, 0.5 M Monobasic Potassium Phosphate and Distilled Water.

Formulation per 100 mL

| | | |
|---|---|---|
| 0.5 M dibasic Potassium Phosphate | 7.50 | mL |
| 0.5 M monobasic Potassium Phosphate | 2.5 | mL |
| Distilled Water | 90.00 | mL |

The above constituent ingredients are then combined in a clean Volumetric Flask. First, in the clean Volumetric Flask, about 80% of distilled water volume is added. Next, about 0.5 M dibasic potassium phosphate is added. Then, about 0.5 M monobasic potassium phosphate is added. The Volumetric Flask with its contents is next placed on the stir plate and the stir bar is added. The contents of the Volumetric Flask are mixed until dissolved. After dissolution, the pH is confirmed as being within the allowed range. QS to final volume with distilled water, cap the receptacle and labeled with the appropriate part and lot number. Store the resulting Buffer at about 2 to about 8° C. The resulting Buffer should be used within 5 days.

25 mM Potassium Phosphate Buffer pH 7.4-7.6

The equipment required for the preparation of 25 mM Potassium Phosphate Buffer pH 7.4-7.6 includes: a Glass Bottle (screw cap), Volumetric Flask, a Stir Plate, a Stir Bar and a pH meter.

The chemicals required: for the preparation of 25 mM Potassium Phosphate Buffer pH 7.4-7.6 includes: 0.5 M Dibasic Potassium Phosphate, 0.5 M Monobasic Potassium Phosphate and Distilled Water.

Formulation per 100 mL

| | | |
|---|---|---|
| 0.5 M K₂HPO₄ | 3.80 | mL |
| 0.5 M KH₂PO₄ | 1.20 | mL |
| Distilled water | 95.00 | mL |

The above constituent ingredients are then combined in a clean Volumetric Flask according to the procedure described above for the 50 mM Potassium Phosphate Buffer pH 7.4- 7.6.

Now, we turn to the preparation of the unique constituent ingredients for the Membrane Blocking Buffers starting with the 0.4% Alkaline Treated Casein Solution.

0.4% Alkaline Treated Casein Solution

The equipment required for the preparation of 0.4% Alkaline Treated Casein Solution includes: a Glass Bottle (screw cap), Measuring cylinder, Balance, a Weigh Boat, a Stir Plate and a Stir Bar.

The chemicals required: for the preparation of 0.4% Alkaline Treated Casein Solution includes: Casein, Boric Acid, Sodium Hydroxide and Distilled Water.

Formulation per 100 mL

| | | |
|---|---|---|
| Casein | 0.400 | gm |
| Sodium hydroxide | 0.053 | gm |
| Boric Acid | 0.260 | gm |
| Distilled water | 100.00 | mL |

The above constituent ingredients are then combined in a clean Volumetric Flask. First, in the clean Volumetric Flask, about 80% of distilled water volume is added. The Volumetric Flask with its contents are next placed on the stir plate and the stir bar is added. Next, NaOH is added. The contents of the Volumetric Flask are mixed until dissolved. After dissolution, the casein is added. The combination is mixed until dissolved, about forty-five (45) minutes. Then the Boric Acid is added and mixed until dissolved. QS to final volume with distilled water, cap the receptacle and labeled with the appropriate part and lot number, date of manufacture and Expiration date. Store the resulting Buffer at about 2 to about 8° C. The resulting casein should be used within 5 days.

The specific manufacturers of the specific ingredients used in the formulations above and the examples below are as follows:

Sigma Casein (C-7078), Sigma Sodium Hydroxide (S-0899), Sigma Boric Acid (B-7660), Sigma Sucrose (S-5016), Pragmatics Surfactant 10G (S-24), Pragmatics Chemal LA-9 (S-22), Aldrich Polyvinyl Alcohol (36,062-7), Sigma Dibasic Potassium Phosphate (P-3786), Sigma Monobasic Potassium Phosphate (P-0662) and Sigma Sodium Azide (S-8032).

Please note that manufacturer, common name and part number is given for each ingredient. It should be understood that the present inventive chemical blocking agents are not limited to these specific ingredients as they are provided as representative of the many possible ingredients that are believed effective to produce results similar to those presented in the present application.

We now turn to actual examples utilizing specific representative formulations of the new, improved chemical blocking agents when applied to nylon membrane.

The hCG Detection Test and Morphine Detection Test example below used the formulation for Chemical Blocking Agent A as the blocking agent.

### EXAMPLE 1

hCG Detection Test

A reinforced microporous membrane for lateral flow applications using the present disclosure was prepared in accordance with Example 1 of the incorporated by reference provisional application. Differences between the membrane of Example 1 of the incorporated by reference provisional application, entitled “Membrane And Method For Making Same With Consistent Wicking Properties For Lateral Flow Assays,” and the lateral flow membrane used as Example 1 below are described below. The reinforced microporous membrane produced was used in a lateral flow sandwich assay for detection of human chorionic gonadotropin (hCG) commonly used in home pregnancy test kit. The reinforced microporous membrane was prepared such that the capillary wicking of the individual strips occurs in the cross web direction.

Preparation of Nylon Membrane Test Strips

The membrane used in the hCG detection test was 99M00106. The membrane was made by exactly the same method as actual Example 1 of the incorporated by reference provisional application, entitled “Membrane And Method For Making Same With Consistent Wicking Properties For Lateral Flow Assays.”

The mother dope comprised about twelve percent (12%) by weight Nylon 66 (Dupont Zytel E53), about eighty-one and four-tenths percent (81.4%) by weight formic acid and about six and six-tenths percent (6.6%) by weight methanol.

The finished lab cast membrane had a thickness of about 8.1 mils, an Initial Bubble Point of about 20.4 psi and a Foam All Over Point of about 22.3 psi. This was evidence that the mother dope, as formulated and produced for this example, had a nominal pore size of about 0.8 microns prior to being processed by a Dial-A-Por™ unit, and further processed into a large pore size microporous nylon membrane by a vertical casting apparatus at a dope processing site.

The specific target temperature for the Dial-A-Por™ unit was about fifty-three and one half degrees Celsius (53.5° C.) to effect a substantially lower bubble point dope attribute, followed by cooling to about twenty-one degrees Celsius (21° C.), to effect a useful dope viscosity for impregnation and coating.

At the dope processing site, a highly uniform non-woven Polyester fiber web or scrim suitable for preparation of the lateral flow membrane (commercially available from Ahlstrom, Inc. Product Grade # Hollytex 3703), having a basis weight of nominally 1.06 oz./sq.yd. (36 gm./sq.meter) was processed by the method taught in the Ser. Nos. 09/040,979 and 09/040,816 applications. The scrim was pre-treated with a mild Corona Discharge to enhance its wettability before being pressure impregnated. The relatively larger pore size dope was provided from the Dial-A-Por™ unit operatively connected to the first slot die (or Membrane Zone One impregnating die) and was used to pressure impregnate the scrim, with an impregnation weight of about ten and nine-tenths grams per square meter (10.9 gm./m²) of nylon solids. The nylon solids are provided from the dissolved nylon in the dope solution, which was, in this example, a twelve percent by weight (12 wt. %) nylon solution (about ninety-one, 91, grams of liquid dope per square meter), which is sufficient to impregnate and fill the void volume of the scrim, and left a small excess of coating dope on the application side of the scrim creating the first zone of large pore size dope integral with the supporting scrim.

The relative amounts of dope per side were adjusted to roughly balance the total coating weight on both sides, and resulted in a finished coating weight of approximately thirty-four (34) grams of nylon solids per square meter (including impregnation weight); therefore, approximately twenty-three and one-tenth (23.1) grams per square meter were distributed between the other two coating slot dies.

The resultant three-zone, geometrically symmetric, pore size symmetric reinforced nylon microporous Lateral flow membrane of this Example had the measured attributes as illustrated in the following Table 1.

TABLE 1

| | FP | Thick | FAOP | IBP | Tₘₐₓ | Wicking Time-CD |
|---|---|---|---|---|---|---|
| Roll | um | Mils | psi | psi | ° C. | sec |
| 99M00106 | 4.306 | 6.3 | 4.7 | 3.5 | 53.5 | 80 |

A monoclonal anti-beta hCG antibody was conjugated to 40 nm gold particles then back-coated with Bovine Serum Albumin plus stabilizing reagents. (The conjugate.)

A mononoclonal anti-alpha hCG antibody was applied to a strip of nylon membrane (30 cm×2.5 cm) using a BioDot™ dispenser to provide a discrete line along the center of the membrane length. (The capture zone.)

The striped nylon membrane was blocked using the Chemical Blocking Agent A solution and then dried at 45° C. The Chemical Blocking Agent A solution used in this example included about 0.15% alkaline treated casein and about 0.10% surfactant 10G.

A sample pad was attached along the top length of the membrane.

An absorbent pad was attached along the bottom length of the membrane.

The membrane was cut to produce 5 mm×2.5 cm test strips. The sample pad located at the beginning of the test strip.

The strip was inserted into a housing such that a sample delivery port was located above the sample pad and a visualization window above the capture zone read-out area.

A series of hCG standards were prepared in PBS containing 2% Bovine Serum Albumin at 1000, 100, 25, 12.5, 6.25 mIU/ml and zero mIU/ml.

Performance of Test

10 μl of conjugate was added to the sample pad.

100 μl standard was added to the sample delivery port in the housing and reagents were allowed to migrate to the terminal end of the membrane.

After the conjugate has passed beyond the visualization window, the results were read. The data are summarized in the table below.

hCG mIU/ml Visual Signal intensity at capture zone

| mIU/ml | Visual | rating |
|---|---|---|
| 1000 | Very Strong | 5 |
| 100 | Strong | 4 |
| 25 | Medium | 3 |
| 12.5 | Weak | 2 |
| 6.25 | Very Weak | 1 |
| 0 | Negative | — |

Any visual color at the capture zone was indicative of the presence of hCG in the sample. As expected, the use of the present inventive combination lateral flow membrane and the chemical blocking agent used in this example of a pregnancy test kit provided superior sensitivity level of 1000 mIU hCG and very good sensitivity at level of 100, 25 and 12.5 mIU hCG in clinical trials. Further, the results at 6.25 mIU hCG were satisfactory, thus demonstrating a superior commercially viable pregnancy test.

### EXAMPLE 2

A reinforced microporous membrane for use in lateral flow IVD applications based on the present disclosure was prepared in accordance with Example 1 above. The reinforced microporous nylon membrane produced was used to produce a lateral flow competitive assay for a morphine detection test kit. The reinforced microporous membrane strips used are oriented such that the capillary wicking of the individual strips was in the cross web direction.

Morphine Detection Test—a Competitive Inhibition Test

The mother dope was identified as Dope #99J086, and comprises about fourteen and five-tenths percent (14.5%) by weight Nylon 66 (Solutia Vydyne 66Z), about seventy-nine and two-tenths percent (79.2%) by weight formic acid and about six and three-tenths percent (6.3%) by weight methanol, was produced by the method disclosed in U.S. Pat. Nos. 3,876,738 and 4,645,602.

The finished lab cast membrane had a thickness of about 8.8 mils, Initial Bubble Point of about 19.6 psi and a Foam All Over Point of about 21.2 psi. This was evidence that the mother dope, as formulated and produced for this example, had a nominal pore size of about 0.8 microns prior to being processed by a Dial-A-Por™ unit, and further processed into large pore size microporous nylon membrane by a vertical casting apparatus at a dope processing site.

The specific target temperature for the Dial-A-Por™ unit was fifty-seven and six tenths degrees Celsius (57.6° C.) to effect a substantially lower bubble point dope attribute, followed by cooling to about twenty-one degrees Celsius (21° C.) to effect a useful dope viscosity for impregnation and coating.

At the dope processing site, a highly uniform non-woven Polyester fiber web or scrim suitable for preparation of the lateral flow membrane (commercially available from Ahlstrom, Inc. Product Grade # Hollytex 3703), having a basis weight of nominally 1.06 oz./sq.yd. (36 gm./sq.meter) was processed by the method taught in the Ser. Nos. 09/040,979 and 09/040,816 applications. The scrim was pre-treated with a mild Corona Discharge to enhance its wettability before being pressure impregnated. The relatively larger pore size dope was provided from the Dial-A-Por™ unit operatively connected to the first slot die (or Membrane Zone One impregnating die) and was used to pressure impregnate the scrim, with an impregnation weight of about ten and nine-tenths grams per square meter (10.9 gm./sq.meter) of nylon solids. The nylon solids are provided from the dissolved nylon in the dope solution. In this example, a fourteen and one half percent by weight (14.5 wt. %) nylon solution (about seventy-five, 75, grams of liquid dope per square meter), which was believed to be sufficient to impregnate and fill the void volume of the scrim, and leave a small excess of coating dope on the application side of the scrim creating the first zone of large pore size dope integral with the supporting scrim.

The relative amounts of dope per side was adjusted to roughly balance the total coating weight on both sides, and resulted in a finished coating weight of approximately thirty-four (34) grams of nylon solids per square meter (including impregnation weight); therefore, approximately twenty-three and one-tenth (23.1) grams per square meter was distributed between the other two coating slot dies.

The resultant three-zone, geometrically symmetric, pore size symmetric reinforced nylon microporous lateral flow membrane of this Example had the measured attributes as illustrated in the following Table 2.

TABLE 2

| | MFP | Thick | FAOP | IBP | Tₘₐₓ | Wicking Time-CD |
|---|---|---|---|---|---|---|
| Roll | um | mils | psi | psi | ° C. | sec |
| 99J08605 | 6.232 | 5.6 | 5.1 | 4.2 | 57.6 | 74.6 |

Preparation of Nylon Membrane Test Strips

A monoclonal anti-morphine antibody was conjugated to gold colloid particles and the conjugate was back-coated with Bovine Serum Albumin plus stabilizing reagents. (The conjugate.)

A soluble morphine/BSA complex was applied to a strip of nylon membrane (30 cm×2.5 cm) using a BioDot™ dispenser to produce a discrete line along the center of the membrane length-perpendicular to the cross-web direction. (The capture zone.)

The striped nylon membrane was blocked using the Chemical Blocking Agent A solution, with the following modifications and then dried for about 40 minutes at about 45° C. The Chemical Blocking Agent A solution used in this example included about 0.15% alkaline treated casein and about 0.20% surfactant 10G.

The test strips were assembled as in Example 1.

A series of morphine standards were prepared in potassium phosphate buffer containing 0.25% BSA at 1000, 100, 20, 10, ng/ml and zero ng/ml.

3 μl conjugate was added to the sample pad.

110 μl standard was added to the sample delivery port of the housing and reagents were allowed to migrate to the terminal end of the membrane.

After conjugate had passed beyond the visualization window, the results were read with the data recorded below.

Morphine ng/ml Visual Signal Intensity

| mIU/ml | Visual | rating |
|---|---|---|
| 1000 | Negative | 0 |
| 100 | Negative | 0 |
| 20 | Negative | 0 |
| 10 | Weak | 2 |
| 0 | Strong | 5 |

No visual signal was indicative of the presence of morphine in the sample at a level greater than 10 ng/ml. A visual signal was indicative of the absence of morphine or the presence of morphine at the minimum level of test sensitivity. In general, for these tests, this signal typically visualized when there was less than about 20 ng/ml in the sample but remain blank past a particular upper threshold; the ability to reliably remain blank against all background noise, at the lowest possible concentration of morphine was the goal of a sensitive test.

With the use of the present inventive combination lateral flow membrane and the inventive chemical blocking agents in a morphine drug-of-abuse test kit provided at least an acceptable sensitivity level of between 10 and 20 ng/ml of morphine in clinical trials, thus demonstrating a commercially useable drug-of-abuse test.

Based on the foregoing, it should be clear that the combination lateral flow membrane and improved chemical blocking agents of the present application solved the need for membranes that can be used more effectively in immunodiagnostic assays for lateral flow IVD applications. The combination lateral flow membrane and improved chemical blocking agents increased the reinforced membrane's wicking rate. The combination lateral flow membrane and improved chemical blocking agents increased the ability of the membrane to detect an analyte of interest at lower levels than those of the prior art. The combination lateral flow membrane and improved chemical blocking agents increased the ability of the membrane to detect an analyte of interest at lower levels.

In the above examples, it has been found that the ratio of nylon volume to chemical blocking agents volume for effective use was full saturation of the membrane void volume, thus, whatever it takes to saturate membrane void volume is operative.

Based on the above examples and the work done to date, the ranges for effective use of the alkaline treated casein (ATC) in the blocking agents of the present application applied to reinforced nylon membrane of the present application included, but was not limited to, about 0.05% to and including about 0.5% alkaline treated casein. It is believed that the alkaline treated casein will function at levels even lower than about 0.05% but will not produce optimal results. It is also believed that the alkaline treated casein will function at a levels as high as 1% or 2% and still have a functional system. Presently, it is believed possible 1% or 2% alkaline treated casein in some cases may be quite functional.

Presently, it is known that blocking agents having concentrations of about 0.15% up to about 0.2% alkaline treated casein as taught in the present application function well. Further, it is believed that all non-specific binding sites in the nylon are saturated at or about a level of about 0.05% alkaline treated casein in the blocking agent.

As stated above, Sigma alkaline treated casein has proved successful in the above applications. However, we expect that comparable casein manufactured by a different company and by a different process would most likely work.

Concerning the surfactants used in the above examples, other surfactants may be optimal for different assays. The surfactants that were used in the above examples have shown themselves to be highly applicable to the morphine test and the pregnancy test but there is no reason to expect that other surfactants like the other surfactants used in various experiments would not also be functional and/or possibly optimized for different assays. Other surfactants that may be operable include, but are not limited to, Sufonyl 465; Pluronic L64; Tetronic 1307; Tween 20 and Triton X-100.

Concerning the concentration range of surfactants, the range of concentrations from about 0.01% to up to about 0.5% was tested. It is known to those skilled in the art that each surfactant will have an optimal range.

Concerning the buffers, the potassium phosphate has proved useful. However, other buffers may also work. Specifically, a buffer known as Trif buffer and one called MOPS buffer are expected to work and would be know to those skilled in the art and, thus, they could also be used.

Concerning determining what is the best buffer to use for any given test, the buffer used must be compatible with the chemistry of the immuno diagnostic test. For example, for a urine test, the buffer should be capable of buffering the test chemistry up to a pH of approximately 7.5.

When testing serum, serum being the remainder after the platelets have been taken out of blood and plasma, as there is a difference between whole blood, plasma and serum and some tests use serum. However, serum is naturally buffered so a strong buffer is not required in the case of serum.

Again, a buffer is used only in order to adjust the pH for optimum binding of the analyte to the test site or capture zone, and can be determined by those skilled in the art.

Concerning the polymer, the polymer operates as a wetting agent and a flow modifier and a protein stabilizer for the reagents on the nylon membrane. The polymer's, polyvinyl alcohol (PVA), primary purpose is as a flow modifier and a protein stabilizer. As tested, the PVA constituent is expected to be functional in the range of about 0.05% up to as high as about 5% of the chemical blocking agent solution, with the presently preferred optimal level being about 0.25%.

Specifically, the polymer, PVA, acts as a flow modifier and protein stabilizer for the colloidal sol, which is the mobile species of the test. In the case of a sandwich style assay, the mobile species is a specific antibody that has been conjugated with a visualization aid such as colloidal gold, or a colored latex bead. When the antigen is present, the colloid labeled specific antibody attaches to the antigen at the epitope to which this antibody is directed, thus forming a antigen-antibody complex. This complex is then the mobile species. Said complex must move along the membrane test strip to the capture zone, where the immobilized antibody can attach and capture the complex, by conjugating to a different epitope of the antigen to which the immobilized antibody is directed. Any un-conjugated colloidal labeled antibody is free to pass by the capture zone (still being a mobile species), to deposit either in an end zone, or in an absorbent pad at the terminal end of the test strip. As described here, it is believed that the PVA polymer stabilizes the colloidal sol as it travels, and assists in the flow transport of the colloidal sol.

Another ingredient used is sucrose. Sucrose is believed to assist in wetting and stabilizing the membrane by helping the blocking solution to wet onto the membrane itself. Thus, it is believed that sucrose has a role in stabilizing the chemical blocking agents on the membrane. Sucrose appears to have a beneficial effect but is believed not to be necessary for the chemical blocking agents to be operative. In other words, it is believed that the chemical blocking agents of the present application will work fine without sucrose.

The last component of the chemical blocking agent is sodium azide which functions as a bacteriostat the purpose of which is to stabilize the finished blocking solution and prevent it from spoilage due to bacteria growth. The range of the bacteriostat is normally about 0.01% up to about 0.1% and a presently referred fictional and useful quantity is about 0.05%. The sodium azide is simply used in the formulation of the chemical blocking agents to stabilize the chemical blocking agents before the chemical blocking agent is applied to the membrane. As soon as the chemical blocking agent is applied to the membrane and the membrane is dried, the sodium azide is driven off in the course of drying the membrane. Therefore, the finished blocked membrane contains no sodium azide when the membrane is being used in a test.

At the end of the above tests, the question arose as to what is the difference between a standard casein solution and the alkaline treated casein solution of the present inventive blocking agent?

After much evaluation of the tests results, several observations could be made about the alkaline treated casein used in the above examples. First, the alkaline treated casein stayed in solution. In other words, the alkaline treated casein did not precipitate out of solution over time. Second, the alkaline treated casein can be used under ambient conditions. In other words, one does not need special conditions to handle or apply or store the alkaline treated casein described in the present application. Further, the preparation of the chemical blocking agent is very simple.

Thus, it appears that the alkaline treatment of the casein stabilizes the casein and modifies the surface of the casein. As regular casein is known to precipitate out a solution after 24 hours, in the sense that regular casein can aggregate and if it aggregates it will precipitate. Thus, it is believed that the alkaline treatment modifies the casein surfaces in such a way as to maintain the particle size and distribution of the casein molecules and enables the casein molecules to interact with the binding sites of nylon in the casein's smallest possible form. Thus, it is further believed that the casein solution is then more effectively blocking the nylon as opposed to if the casein were clumping or aggregating. This clumping or aggregating is believed to cause the casein to be unable to effectively treat the surface of the nylon.

Concerning why alkaline treated casein works better than regular untreated casein, it is also believed now that the alkaline treatment prevents aggregation of casein during the drying process when the chemical blocking agent solution is dried onto the nylon membrane. So if aggregation is prevented during the drying process, the alkaline treated casein is rendered more effective as a blocker. Again, along the same lines as above, it is believe that, if the casein particles are kept small, they interact on a smaller more molecular level with the functional groups of nylon membrane which need chemical blocking.

While the application of the inventive chemical blocking agents have been proven, as demonstrated above, it is believed that the inventive chemical blocking agents will be effective as a blocking agents for other nylon membrane suitable for use in immunodiagnostic assays, such as, for example, nylon membrane used in flow-through format type applications and in Nucleic Acid Detection Assays. Toward that end, the following prophetic examples are presented.

### EXAMPLE 3 (PROPHETIC)

Pregnancy Test Kits in Flow-through Format Using Nylon Membrane Support and Improved Chemical Blocking Agent

As mentioned in the background of the disclosure, another useful format for producing in vitro diagnostic test kit utilizes membranes and blocking chemistries or chemical blocking agents in the flow-through mode, as opposed to the lateral flow mode assays addressed above. Flow-through assays can be produced for a variety of diagnostic tests, using reagents similar to those used in lateral flow assays. Both “sandwich” assays and “competitive” assays can be successfully designed. Flow-through assays are generally multiple step procedures, involving more operator interaction, when compared to lateral flow assays. Nylon would be a preferred substrate for these test assays, due to the natural hydrophilicity of nylon, and nylon's ability to selectively immobilize one or more of a variety of tests components as needed, if non-specific binding locations could be satisfactorily blocked.

When used in conjunction with the present inventive chemical blocking agent formulations, it is believed that nylon membrane can be treated at the appropriate step to effectively reduce or eliminate non-specific binding, providing tangible improvements such as greater sensitivity to low analyte concentration and higher resolution in these tests. The use of nylon with the present inventive chemical blocking agent formulations may simplify these type of tests, reducing the number of steps needed to achieve an end-point.

Concerning the nylon membrane substrate used in flow-through type tests, it is not important to have uniform lateral wicking rates; it is more important to have uniform and controlled trans-membrane flow-through attributes, which are largely controlled by the uniformity of pore size and the selection of reinforcements which are uniform and non-restrictive to trans-membrane flow rates. In other words, a more traditional nylon microporous filtration membrane having a pore size and pore distribution optimized for trans-membrane wicking flow rate is more likely to be useful than one optimized for lateral flow attributes. In this prophetic example, the use of the chemical blocking agents of the present disclosure, optimized for nylon membrane, should enhance the performance and sensitivity, and potentially reduce the complexity of the flow-through membrane assay.

In the flow-through mode, in the example of sandwich type assay, the primary antibody may be immobilized directly to the surface on the nylon membrane, or indirectly by immobilization on a latex bead, the bead is then attached to the surface of the membrane by mechanical or other capture means. The primary antibody or immobilization matrix is applied to the surface in a recognizable pattern (such as a line, or a dot, or other pattern), so that the presence of the analyte is determined by a visual discrimination of the pattern against the background color of the membrane.

Other patterns may also be pre-printed on the nylon membrane, to allow a second and/or third visualization in response to particular chemistries of the assay to indicate positive and/or negative controls to the test. For example, a positive test control line of hCG antigen can be printed crosswise to the primary antibody line, such that the finished assay will show a “plus” sign if the analyte is present in the test sample, and a “minus” sign if no analyte is present in the test sample.

In the test assay, the analyte (being the antigen, or in the case of a pregnancy test, the hCG hormone) is introduced to the primary antibody by pouring a predetermined amount of test sample or analyte carrier (example, urine) directly onto the surface of the membrane, and allowing the carrier to soak through from top side to bottom side in a trans-membrane flow direction (as opposed to the lateral flow direction of examples 1 and 2). The trans-membrane flow is assisted generally by containing the membrane in a small housing, where the exposed top side of the membrane is the surface to which the antibody or other detection means has been applied, and the bottom side is in communication with a receptacle well, capable of absorbing the fluid which traverses the membrane. Such a receptacle will contain layers of absorbent material directly contacting the bottom side of the membrane.

The analyte (example, hCG), if present, will bind to the immobilized primary antibody, which attaches to an epitope of the hCG to which the primary antibody is directed. Since the primary antibody has been applied to the top-side of the membrane, this will be the visualization surface.

The carrier may have been pre-mixed with a detection system (such as a secondary antibody conjugated to a gold colloid, or other visualization aid). Alternatively, the carrier may be the pure substance (urine), and the detection system is added in a second step. In either case, the secondary antibody has been pre-conjugated with a suitable visual detection agent, or portion of a visual detection agent.

The secondary antibody will attach to a different epitope of the hCG to which the secondary antibody is directed. When the conjugated antibody plus visual detection aid is present at any particular spot on the surface of the membrane in a high enough concentration, the color of the visual detection aid is intended to be more pronounced and easily discriminated against the background color of the membrane.

Thus, it is important that the binding occur specifically at the intended positions and patterns previously mentioned, and not on portions of the membrane surface which are not part of these patterns. Such unintended binding will decrease the ability to visually resolve the pattern, by increasing visual noise or background. This might lead to a false negative. Alternatively, if unintended binding of conjugate was to occur directly over the immobilized antibody pattern in the absence of true signal antigen, this could lead to a false positive interpretation.

In practice, there are wash steps between applications. These steps are intended to ensure that the specific binding has taken place, and minimize the chance of residue of unbound conjugate or unbound analyte being left on any portion the surface. Also, there may be separate steps having further additive chemistries applied, to amplify or complete the visualization.

Since this is a multi-step process, there is an expanded set of roles chemical blocking agent. The chemical blocking agent formulation may be applied to the membrane after spotting the primary antibody (and/or other pattern generating moieties) directly onto the nylon membrane. Alternatively, the chemical blocking agent formulation may be applied to the membrane before immobilizing the (primary antibody-treated) latex beads.

In either case, the first role of the chemical blocking agent is to prevent non-specific binding of the analyte of interest. (The term non-specific binding as used here is intended to describe the binding of any test component to the portions of the nylon membrane surface which are not intended for pattern visualization and analysis, but are intended to provide a blank background against which the patterns can be discerned.)

The second role of the chemical blocking agent is to prevent non-specific binding of the secondary antibody that is conjugated to the visualization agent. Alternatively, if the carrier has been pre-mixed with the detection system, the second role of the chemical blocking agent is to prevent non-specific binding of the complex of the antigen-plus-secondary (conjugated) antibody.

If further wash steps are needed, the third role of the blocking formulation is to enhance the efficiency of these steps, minimizing the total volume of wash required to clear the visualization surface of the unbound test component.

If signal amplification is needed, the fourth role of the chemical blocking agent formulation is to prevent non-specific absorption of such signal-generating or amplifying species.

Such flow-through in vitro diagnostic tests are known in the art. One such system is produced by Hybritech, Inc; as the ICON® II hCG (Urine) test system, which is commonly used in hospital laboratories.

In the present prophetic example 3, an improved chemical blocking agent formulation such as one of the chemical blocking agent formulations described above, when applied to a flow-through nylon membrane of suitable pore size and distribution, will generally be a more effective chemical blocking agent, than prior blocking agents, effectively reducing non-specific binding in the first role.

For those flow-through assays, which require a rinse buffer following analyte application, the use of chemical blocking agent formulations described above, will minimize the volume of a “chaser” wash step to clear the surface of the nylon membrane. It is also expected that the use of the improved blocking formulation may completely eliminate the need for such a “chaser” wash step.

Furthermore, in the present prophetic example, it is expected that the blocking formulation will more effectively prevent non-specific binding of the secondary antibody that is conjugated to the visualization agent. Thus, the conjugate will link exclusively to the specific epitope of the hCG to which the secondary antibody is directed.

Furthermore, in the present prophetic example, it is expected that the blocking formulation will decrease the total volume of rinse buffer necessary in the third role to clear the visualization surface of the membrane of all un-bound conjugate.

Furthermore, in the present prophetic example, if there is to be a signal amplification step, it is expected that the blocking formulation will again more effectively prevent non-specific binding of the signal amplifier to any area outside of the complexed conjugate which is, by this step, specifically bound in the desired pattern.

The net result of using the present inventive blocking formulations in the nylon based flow through assay is to realize a more sensitive and accurate test, in light of the combination of improvements discussed here. By combining the improvements in each of the above steps, there is expected to be an overall decrease in background noise, and therefore higher visual clarity of the pattern signal, thus enabling detection at lower concentrations of analyte, and reduced risk of false negatives. Furthermore, the amounts of rinse buffers needed to clear the surfaces is expected to be reduced, and offer the possibility of elimination of one or more of these steps Finally, it is expected that, in light of the improved sensitivity and accuracy offered herein, it will be possible to develop improved quantitative assays, as opposed to strictly qualitative “Yes/No” assays. For example, an immunodiagnostic assay kit could be developed which allows quantification of analyte by correlating the strength of the signal that develops in the capture zone to a calibration chart of signal strength versus concentration of analyte. The calibration chart would be built on a continuum of known reference concentrations which, by prior testing, result in a continuum of output signal strengths. Such a system could be automated, with a small hand-held device containing minimum spectrophotometric optics and a means to interpret the resulting signal against such a calibration chart. A model for such a system would be a spectrophotometric based glucose meter, commonly used by diabetics for the measurement of glucose level in whole blood. Such systems are common, and many utilize microporous membranes impregnated with color generating chemistry, the strength of color being correlated to the concentration of glucose. Generally, the color change, which results from the test, is expressed in a particular reading zone. The reading zone is scanned with minimal optics, and the color is automatically translated into a glucose reading by comparison to the pre-programmed programmed calibration chart. Such a system can be readily developed by one skilled in the art of hand-held instrument manufacture.

Use of Blocking Agent in Nucleic Acid Detection Assays

Concerning the use of the inventive chemical blocking agent formulations in Nucleic Acid Detection Assay applications, it is believed that the chemical blocking agent formulations of the present application will be most useful for Nucleic Acid Detection Assay applications.

The transfer of nucleic acids (DNA and/or RNA) to nylon membrane supports after resolution through agarose or acrylamide gels as a common method for gene discovery analysis was discussed in the background of the disclosure.

In the above methods, detection of the nucleic acids bound to the solid support was achieved either directly or indirectly, as described in the background of the present application. The proposed blocking reagent will allow for the use of nylon membranes having a variety of positive charge densities to be used in chemiluminescent and fluorescent-based detection methods. It is believed that the chemical blocking agent formulations herein described are more broadly effective in preventing non-specific binding of both the modified probe (or target molecule) and the detection/visualization reagent utilized to develop the signal by reacting with or binding to the nylon membrane preventing non-specific binding of the modified probe to the membrane, whereby allowing the modified probe to hybridize to the membrane-bound target. It is expected that the chemical blocking agent formulations of the present application will also be generally more effective than presently available chemical blocking agent formulations. Therefore, it is expected that a higher, more favorable signal-to-noise ratio will be realized. Examples of the results expected when the chemical blocking agent formulations are used for Nucleic Acid Detection Assay applications are described below.

### EXAMPLE 4 (PROPHETIC)

Detection of membrane-bound nucleic acid by direct labeling.

The nucleic acid is fixed following Southern/Northern transfer or spotting on the nylon membrane. The nylon membrane containing the bound nucleic acid is transferred to a plastic tray with a lid, to a glass hybridization bottle with a water-tight seal, or to a sealable plastic bag. In the case of nylon membrane on glass slides, the slides can be placed in a hybridization chamber (Genomic Solutions™ GeneTAC Hybridization Station) or a 50 mL conical tube. The appropriate volume (about 0.125-0.25 mL/cm² of membrane) of the inventive chemical blocking agent is added and the nylon membrane is incubated at a set temperature from about 30 minutes to about several hours. This step is believed necessary for blocking non-specific binding sites on the nylon membrane, ensuring that the labeled probe hybridize only to its target sequence not to the nylon membrane. In comparison to isotopically-labeled probes (³²P, ³³P), probes (or targets) containing fluorescently labeled nucleotides are especially “sticky” (Cy3, Cy5, and BODIPY, structures of which are known in the art) and increased background signal results from their binding non-specifically to solid supports.

After the non-specific binding sites on the nylon membrane, or nylon membrane-coated slide have been made inaccessible with the inventive chemical blocking agent, the labeled probe molecule is added to the hybridization vessel and the reaction is allowed to proceed overnight.

The next day the nylon membranes are exposed to a series of stringency washes. These washes use increasing temperature and decreasing ionic strength to eliminate any non-specific hybrids that have formed between the membrane-bound DNA sequences not completely complementary to the labeled probe (or target). Upon completion of these washes, the signal from the hybrids formed between target and probe is detected using autoradiography or phosphorimaging screens for isotopic-based labeling, or fluorescent imaging systems for fluorophore-based labeling.

### EXAMPLE 5 (PROPHETIC)

Indirect Detection of Membrane-bound Nucleic Acids

The nucleic acid is fixed following Southern/Northern transfer or spotting of the nucleic acid to the nylon membrane, as described above. The nylon membrane containing the bound nucleic acid is transferred to either a plastic tray with a lid, a glass hybridization bottle with a water-tight seal, or a sealable plastic bag. In the case of membrane on glass slides, the slides can be placed in a hybridization chamber (Genomic Solutions™ GeneTAC Hybridization Station) or a 50 mL conical tube. The inventive chemical blocking agent is added to the vessel as described in Prophetic Example 4 and the nylon membrane is prehybridized at a predetermined temperature from about 30 minutes to about several hours. Following the prehybridization a probe (or target, in the case of a microarray) containing steroid hapten molecules, (for example, biotin or digoxygenin), or coupled enzyme is added to the hybridization mixture. The reaction is allowed to proceed overnight. The next day the nylon membranes are exposed to a series of stringency washes to dissociate hybrid molecules that contain imperfect matches between probe and target, as described above.

Indirect assay methods actually detect the PRESENCE of the newly formed hybrid molecule not simply its formation, as is the case with the direct label methods. In order to detect this molecule, the nylon membrane or membrane coated glass slide is incubated with a fluorescently tagged protein, antibody, or a chemiluminscent substrate (enzyme conjugated probe molecule). In the case of biotin, a fluorophore-conjugated streptavidin molecule is used to detect the biotinylated molecule: membrane bound nucleic acid hybrid.

For this type of detection, non-specific binding of the antibody or chemiluminescent substrate must be reduced or eliminated. Incubation of the nylon membrane containing the target: probe hybrid molecule prior to addition of the detection reagents in inventive chemical blocking agent is expected to prevent the non-specific binding mentioned above. The inventive chemical blocking agent is added to the membrane and allowed to incubate at room temperature with gentle agitation minimally for about 60 minutes. The solution is drained away and the detection reagent is added as described in the Manufacturer's Instructions. The signal generated is detected using a fluorescent detector, CCD Camera System for chemiluminsecence, or with autoradiography film.

The composition of the chemical blocking agents herein described is more broadly effective in preventing non-specific binding of both the modified probe (or target molecule) and the detection/visualization reagent utilized to develop the signal by reacting with or binding to the modified probe bound to the membrane-bound target. Thus, it is expected that the chemical blocking agents of the present disclosure will be generally more effective than the presently available blocking formulations. Therefore, it is expected that a higher, more favorable signal-to-noise ratio will be realized when using the inventive chemical blocking agents of the present application for Nucleic Acid Detection Assay applications.

While the compositions, articles and methods of using and making the articles and compositions described herein constitute preferred embodiments of the disclosure, it is to be understood that the disclosure is not limited to these precise compositions, articles and methods and that changes may be made therein without departing from the scope of the disclosure which is defined in the appended claims.

What is claimed is:

1. A chemical blocking agent for use with nylon membrane suitable for use in assays comprising:
an effective amount of alkaline treated casein;
an effective amount of polymer; and
an effective amount of surfactant.

2. The chemical blocking agent of claim 1 further comprising:
an effective amount of stabilizer.

3. The chemical blocking agent of claim 2, wherein the stabilizer is sodium azide.

4. The chemical blocking agent of claim 1, wherein the assay is selected for the group comprising:
immunodiagnostic assays, flow-through assays and nucleic acid detection assays.

5. The chemical blocking agent of claim 1, wherein the amount of alkaline treated casein is from about 0.05% to about 2.0%.

6. The chemical blocking agent of claim 1, wherein the amount of alkaline treated casein is from about 0.1 to about 1.0%.

7. The chemical blocking agent of claim 1, wherein the amount of alkaline treated casein is from about 0.15 to about 0.2%.

8. The chemical blocking agent of claim 1, wherein the amount of polymer is from about 0.05% up to about 5%.

9. The chemical blocking agent of claim 1, wherein the amount of polymer is about 0.25%.

10. The chemical blocking agent of claim 1, wherein the amount of surfactant is from about 0.01% to about 0.5%.

11. The chemical blocking agent of claim 10, wherein the amount of surfactant is about 0.05%.

12. The chemical blocking agent of claim 1 further comprising:
an effective amount of Sucrose.

13. The chemical blocking agent of claim 12, wherein the effective amount of Sucrose is about 0.015%.

14. A nylon microporous membrane suitable for use in assays comprising:
a nylon membrane formed from a dope; and
an effective amount of a blocking agent, operatively distributed throughout the nylon membrane.

15. The nylon microporous membrane of claim 14, wherein the chemical blocking agent comprises:
an effective amount of alkaline treated casein;
an effective amount of polymer; and
an effective amount of surfactant.

16. The chemical blocking agent of claim 15 further comprising:
an effective amount of stabilizer.

17. The chemical blocking agent of claim 16, wherein the stabilizer is sodium azid.

18. The chemical blocking agent of claim 15, wherein the assay is selected for the group comprising:
immunodiagnostic assays, flow-through assays and nucleic acid detection assays.

19. The chemical blocking agent of claim 15 further comprising:
an effective amount of Sucrose.

20. The chemical blocking agent of claim 19, wherein the effective amount of Sucrose is about 0.015%.

21. The nylon microporous membrane of claim 14 further comprising:
a porous scrim substantially impregnated by at least a first dope to form a membrane having two sides, the membrane having the porous scrim encapsulated therein.

22. The chemical blocking agent of claim 15, wherein the amount of alkaline treated casein is from about 0.05% to about 2.0%.

23. The chemical blocking agent of claim 15 wherein the amount of alkaline treated casein is from about 0.1 to about 1.0%.

24. The chemical blocking agent of claim 15, wherein the amount of alkaline treated casein is from about 0.15 to about 0.2%.

25. The chemical blocking agent of claim 15 wherein the amount of polymer is from about 0.05% up to about 5%.

26. The chemical blocking agent of claim 15 wherein the amount of polymer is about 0.25%.

27. The chemical blocking agent of claim 15, wherein amount of surfactant is from about from about 0.05%.

28. The chemical blocking agent of claim 15, wherein amount of surfactant is from about from about 0.01% to about 0.5%.

29. The chemical blocking agent of claim 15 further comprising:
an effective amount of Boric acid.

30. The chemical blocking agent of claim 29 wherein the effective amount of Boric acid is about 0.12%.

31. A method of preparing a chemical blocking agent for use with nylon membrane suitable for use in assays comprising the acts of:
mixing an effective amount of potassium phosphate buffer and an effective amount of alkaline treated casein in a receptacle;
adding an effective amount of surfactant;
diffusing the surfactant into solution;
once in solution, adding an effective amount of sucrose;
dissolving the effective amount of sucrose;
once the sucrose is dissolved, adding an effective amount of polymer;
allowing the effective amount of polymer to dissolve for about sixty (60) minutes;
upon expiration of the sixty (60) minutes, filtering the solution through an about 0.2 um filtration device into a receptacle.

32. The chemical blocking agent of claim 31, wherein the assay is selected for the group comprising:
immunodiagnostic assays, flow-through assays and nucleic acid detection assays.

33. The method of claim 31, wherein the effective amount of potassium phosphate buffer comprises:
about 50 mM potassium phosphate buffer; and
about 25 mM potassium buffer.

34. The method of claim 31, wherein the chemical blocking agent further comprises:
an effective amount of stabilizer.

35. The method of claim 31, wherein, the chemical blocking agent further comprises:
an effective amount of Boric acid.

36. The method of claim 31, wherein the chemical blocking agent further comprises:
an effective amount of Sucrose.

37. The method of claim 31, wherein the effective amount of Sucrose is about 0.015%.

38. A method of using the membrane of claim 14 to detect an analyte of interest comprising:
contacting the membrane with a fluid believed to contain the analyte of interest; and
detecting the analyte of interest if present in the fluid.

39. A method of using the membrane of claim 14 to detect a analyte of interest comprising:
contacting the membrane with a fluid comprising the analyte of interest; and
detecting the analyte of interest on the membrane.

40. An immunodiagnostic assay kit comprising the membrane of claim 14 and a means for detecting an analyte of interest.

41. An immunodiagnostic assay kit comprising the membrane of claim 14 and a means for quantifying an analyte of interest by correlating the strength of the signal to a known reference concentration continuum which is correlated to a continuum of signal strengths.
